SEQUENCE LISTING (46 sequences)

SEQ ID NO: 1 - DNA, 21 bp, Artificial Sequence
  Forward primer for amplifying AFP
agactgctgcagccaaagtga

SEQ ID NO: 2 - DNA, 20 bp, Artificial Sequence
  Reverse primer for amplifying AFP
gtgggatcgatgctggagtg

SEQ ID NO: 3 - DNA, 20 bp, Artificial Sequence
  Forward primer for amplifying SOX9
gaggaagtcggtgaagaacg

SEQ ID NO: 4 - DNA, 20 bp, Artificial Sequence
  Reverse primer for amplifying SOX9
atcgaaggtctcgatgttgg

SEQ ID NO: 5 - DNA, 20 bp, Artificial Sequence
  Forward primer for amplifying EpCAM
gaacaatgatgggctttatg

SEQ ID NO: 6 - DNA, 20 bp, Artificial Sequence
  Reverse primer for amplifying EpCAM
tgagaattcaggtgcttttt

SEQ ID NO: 7 - DNA, 20 bp, Artificial Sequence
  Forward primer for amplifying FOXJ1
cctgtcggccatctacaagt

SEQ ID NO: 8 - DNA, 19 bp, Artificial Sequence
  Reverse primer for amplifying FOXJ1
agacaggttgtggcggatt

SEQ ID NO: 9 - DNA, 20 bp, Artificial Sequence
  Forward primer for amplifying CD44
catctaccccagcaacccta

SEQ ID NO: 10 - DNA, 20 bp, Artificial Sequence
  Reverse primer for amplifying CD44
ctgtctgtgctgtcggtgat

SEQ ID NO: 11 - DNA, 22 bp, Artificial Sequence
  Forward primer for amplifying ITGA6
tcgctgggatcttgatgcttgc

SEQ ID NO: 12 - DNA, 24 bp, Artificial Sequence
  Reverse primer for amplifying ITGA6
tgagcatggatctcagccttgtga

SEQ ID NO: 13 - DNA, 22 bp, Artificial Sequence
  Forward primer for amplifying HNF6
agggtgctctgccgctcccagg

SEQ ID NO: 14 - DNA, 24 bp, Artificial Sequence
  Reverse primer for amplifying HNF6
catgctgctaagcggagcgcggac

SEQ ID NO: 15 - DNA, 20 bp, Artificial Sequence
  Forward primer for amplifying HNF1beta
ggggcccgcgtcccagcaaa

SEQ ID NO: 16 - DNA, 21 bp, Artificial Sequence
  Reverse primer for amplifying HNF1beta
ggccgtgggctttggaggggg

SEQ ID NO: 17 - DNA, 20 bp, Artificial Sequence
  Forward primer for amplifying CK19
tccgaaccaagtttgagacg

SEQ ID NO: 18 - DNA, 20 bp, Artificial Sequence
  Reverse primer for amplifying CK19
ccctcagcgtactgatttcc

SEQ ID NO: 19 - DNA, 20 bp, Artificial Sequence
  Forward primer for amplifying CD90
ctagtggaccagagccttcg

SEQ ID NO: 20 - DNA, 20 bp, Artificial Sequence
  Reverse primer for amplifying CD90
acagggacatgaaatccgtg

SEQ ID NO: 21 - DNA, 20 bp, Artificial Sequence
  Forward primer for amplifying CK7
gataaaaggcgcggagtgtc

SEQ ID NO: 22 - DNA, 20 bp, Artificial Sequence
  Reverse primer for amplifying CK7
gaaaccgcaccttgtcgatg

SEQ ID NO: 23 - DNA, 20 bp, Artificial Sequence
  Forward primer for amplifying CFTR
agttgcagatgaggttgggc

SEQ ID NO: 24 - DNA, 20 bp, Artificial Sequence
  Reverse primer for amplifying CFTR
aaagagcttcaccctgtcgg

SEQ ID NO: 25 - DNA, 21 bp, Artificial Sequence
  Forward primer for amplifying AE2
ggggaaagttgagttgggaga

SEQ ID NO: 26 - DNA, 20 bp, Artificial Sequence
  Reverse primer for amplifying AE2
ctggctctggcgtacagaaa

SEQ ID NO: 27 - DNA, 21 bp, Artificial Sequence
  Forward primer for amplifying AQPR1
ggccagcgagttcaagaagaa

SEQ ID NO: 28 - DNA, 21 bp, Artificial Sequence
  Reverse primer for amplifying AQPR1
tcacaccatcagccaggtcat

SEQ ID NO: 29 - DNA, 22 bp, Artificial Sequence
  Forward primer for amplifying Alb
cacagaatccttggrgaacagg

SEQ ID NO: 30 - DNA, 22 bp, Artificial Sequence
  Reverse primer for amplifying Alb
atggaaggtgaatgtttcagca

SEQ ID NO: 31 - DNA, 20 bp, Artificial Sequence
  Forward primer for amplifying HNF1alpha
agagaggggtgtccccatca

SEQ ID NO: 32 - DNA, 20 bp, Artificial Sequence
  Reverse primer for amplifying HNF1alpha
cttgtgccggaaggcttctt

SEQ ID NO: 33 - DNA, 20 bp, Artificial Sequence
  Forward primer for amplifying HNF4alpha
ccaaaaccctcgtcgacatg

SEQ ID NO: 34 - DNA, 20 bp, Artificial Sequence
  Reverse primer for amplifying HNF4alpha
gcacattctcaaattccagg

SEQ ID NO: 35 - DNA, 20 bp, Artificial Sequence
  Forward primer for amplifying ASGR1
cagcaacttcacagccagca

SEQ ID NO: 36 - DNA, 20 bp, Artificial Sequence
  Reverse primer for amplifying ASGR1
agctgggactctagcgactt

SEQ ID NO: 37 - DNA, 20 bp, Artificial Sequence
  Forward primer for amplifying CYP1A2
cggacagcacttccctgaga

SEQ ID NO: 38 - DNA, 20 bp, Artificial Sequence
  Reverse primer for amplifying CYP1A2
aggcaggtagcgaaggatgg

SEQ ID NO: 39 - DNA, 18 bp, Artificial Sequence
  Forward primer for amplifying CYP2A6
cagcacttcctgaatgag

SEQ ID NO: 40 - DNA, 23 bp, Artificial Sequence
  Reverse primer for amplifying CYP2A6
aggtgactgggaggacttgaggc

SEQ ID NO: 41 - DNA, 22 bp, Artificial Sequence
  Forward primer for amplifying CYP2C9
ctacagataggtattaaggaca

SEQ ID NO: 42 - DNA, 23 bp, Artificial Sequence
  Reverse primer for amplifying CYP2C9
gcttcatatccatgcagcaccac

SEQ ID NO: 43 - DNA, 23 bp, Artificial Sequence
  Forward primer for amplifying CYP3A4
ttcagcaagaagaacaaggacaa

SEQ ID NO: 44 - DNA, 22 bp, Artificial Sequence
  Reverse primer for amplifying CYP3A4
ggttgaagaagtcctcctaagc

SEQ ID NO: 45 - DNA, 20 bp, Artificial Sequence
  Forward primer for amplifying GAPDH
tgcaccaccaactgcttagc

SEQ ID NO: 46 - DNA, 21 bp, Artificial Sequence
  Reverse primer for amplifying GAPDH
ggcatggactgtggtcatgag

(19) United States
(12) Patent Application Publication
(10) Pub. No.: US 2020/0147143 A1
(43) Pub. Date: May 14, 2020

(54) HUMAN ADULT HEPATOCYTE REPROGRAMMING MEDIUM COMPOSITION

(71) Applicant: INDUSTRY-UNIVERSITY COOPERATION FOUNDATION HANYANG UNIVERSITY, Seoul (KR)

(72) Inventors: Dongho CHOI, Seoul (KR); Jaemin JEONG, Seoul (KR); Yohan KIM, Seoul (KR); Kyojin KANG, Seoul (KR)

(21) Appl. No.: 16/618,270

(22) PCT Filed: May 28, 2018
(86) PCT No.: PCT/KR2018/006015

§ 371 Date: Nov. 29, 2019

(30) Foreign Application Priority Data
May 29, 2017 (KR) 10-2017-0066120

(51) Int. Cl. A61K 35/407; C12N 5/071

(52) U.S. Cl. CPC A61K 35/407; C12N 5/067; C12N 2501/999; C12N 2501/12; C12N 2506/14

(57) ABSTRACT

The present invention relates to a human adult hepatocyte reprogramming medium composition and provides a method for inducing hepatic progenitor cells from human adult hepatocytes by means of a combination of chemicals.

### BACKGROUND

### 1. Field of the Invention

The present invention relates to a medium composition for reprogramming human adult hepatocytes to hepatic progenitor cells.

### 2. Discussion of Related Art

Liver transplantation is the only treatment that can cure chronic liver disease. Regenerative medicine is one of the most available breakthrough technologies to overcome these problems. Recently, specific stem cells of a patient can be regarded as a reliable source for liver regenerative medicine. Although stem cell-like hepatocytes have been derived from induced pluripotent stem cells (iPSCs), direct reprogrammed cells, and small hepatocytes, none of them show definitive treatment potential for chronic liver disease. Recently, hepatic progenitors induced by chemical derived from rodent represent a potential source for developing cell therapies and drug testing for various kinds of liver disease.

Since orthotropic liver transplantation has been approved as one of the treatment options for end stage liver disease, surgical outcome of liver transplantation improved dramatically and it has been accepted as a standard treatment of choice for various kinds of terminal stage liver disease. There are thousands of liver transplants are done in a year and many patients have to wait for organ donation for a long time and fail to obtain fresh organs or hepatocytes from donors. In terms of new hepatocytes, recent advances in stem cell research have shown promising potential for cell replacement therapy. However, primary hepatocytes are easily apoptotic and difficult to handle and expand in vitro culture conditions, and the establishment of culture conditions of functionally proliferating hepatocytes is an essential part of liver regenerative medicine.

Even though many researchers have reported human embryonic stem cells (hESCs) and induced pluripotent stem cells (iPSCs) as alternative cell sources of hepatocytes, these cells have some limitations. Hepatocytes directly converted from terminally differentiated cells are recent developed cell sources and express hepatocyte specific marker genes and proteins. They are relatively primitive and need more manipulations. Also, many researchers are looking for hepatic progenitors/stem cells to overcome these problems. So far, several studies have suggested that hepatocyte/stem cells can be derived from mice and rats. Furthermore, some papers indicate that a combination of several small molecules, particularly, a combination of Y-27632, A83-01, and CHIR99021 induces hepatic progenitor/stem cells from mouse and rat hepatocytes, but this has not been established in human adult hepatocytes.

### SUMMARY OF THE INVENTION

An object of the present invention is to provide a medium composition for reprogramming human adult hepatocytes to hepatic progenitor cells by adding various chemicals to human adult hepatocytes.

Another object of the present invention is to provide a method for reprogramming human adult hepatocytes to hepatic progenitor cells by culturing human adult hepatocytes in the medium composition for reprogramming.

Still another object of the present invention is to provide hepatic progenitor cells induced through the reprogramming and a composition for treating liver disease including the hepatic progenitor cells.

Yet another object of the present invention is to provide a method for treating liver disease, wherein the method includes administering an effective amount of the hepatic progenitor cells to a subject in need thereof.

In order to achieve the objects, the present invention provides a medium composition for reprogramming human adult hepatocytes to hepatic progenitor cells, including HGF, A83-01, and CHIR99021.

The present invention also provides a method for reprogramming human adult hepatocytes to hepatic progenitor cells, wherein the method includes culturing human adult hepatocytes in a medium composition for reprogramming human adult hepatocytes to hepatic progenitor cells, including HGF, A83-01, and CHIR99021.

The present invention also provides hepatic progenitor cells derived from human adult hepatocytes, having 1.5-fold increased expression of hepatic progenitor cells specific marker including albumin, AFP, SOX9, ITGA6, HNF6, EpCAM, FOXJ1, HNF1β, CK19, CD44, and CD90 compared to that in human adult hepatocytes, and having bipotent stemness differentiating into hepatocytes and biliary epithelial cells.

The present invention also provides a composition for treating liver disease, including the hepatic progenitor cells.

The present invention also provides a method for treating liver disease, wherein the method includes administering an effective amount of a composition for treating liver disease, including hepatic progenitor cells to a subject in need thereof.

The present invention reprograms human adult hepatocytes to hepatic progenitor cells by adding various chemical combinations to human adult hepatocytes and can be used for the treatment of liver disease because the hepatic progenitor cells have a differentiation potential of differentiating into hepatocytes and biliary epithelial cells, and thus can be provided as a source of hepatic precursor for liver regeneration.

### BRIEF DESCRIPTION OF THE DRAWINGS

FIG. 1 illustrates a method of preparing hepatic progenitor cells chemically derived from the human hepatocytes of the present invention.

FIG. 2A illustrates a morphological change (a) of human hepatocytes cultured under AC(+)and AC(−) conditions including HGF for 14 days in the cell differentiation procedure.

FIG. 2B illustrates a growth curve of human hepatocytes under AC(+) and AC(−) conditions from day 0 to day 15 (in the figure, A is an abbreviation for A83-01, C is an abbreviation for CHIR99021. Scale bars=500 μm and 100 μm) (Data is represented as the mean±SD, the culture medium is supplemented with HGF).

FIG. 2C illustrates an immunofluorescence analysis result of hepatic progenitor markers in human hepatocytes cultured under an AC condition for 14 days (Scale bars=50 μm).

FIG. 2D illustrates a quantitative comparison result of the relative gene expression levels of hepatic progenitor markers in human hepatocytes cultured under AC(+) and AC(−) conditions (Data is represented as the mean±SD).

FIG. 2E illustrates a karyotype image analysis result of human hepatocytes cultured under the AC condition.

FIG. 3A illustrates a time lapse imaging result of human hepatocytes cultured under the AC condition including HGF for 9 days.

FIG. 3B illustrates a morphological change of human hepatocytes cultured with chemicals (Y27632, A83-01, and CHIR99021) (in the figure, Y is abbreviation for Y27632, A is abbreviation for A83-01, and C is abbreviation for CHIR99021. Scale bars=100 μm).

FIG. 3C illustrates a morphological change of mouse hepatocytes cultured under AC(+) and AC(−) conditions including HGF (Scale bars=100 μm). FIG. 3D illustrates a morphological change of human hepatocytes under AC or A, C only under HGF (+) and HGF (−) conditions (scale bars=100 μm).

FIG. 4A illustrates the bipotent differentiation potential of CdH into hepatocytes and bile duct epithelium, in vitro hepatic differentiation of CdH (Arrow indicates bile canaliculi), analysis of glycogen storage by Periodic Acid Schiff (PAS) staining, Indocyanine Green (ICG) uptake analysis (Scale bars=100 μm), the immunofluorescence analysis result of hepatic markers in hepatic-differentiated CdH (CdH-Hep) (Scale bars=50 μm).

FIG. 4B is a quantitative comparison result of the gene expression levels of hepatic markers for CdH, hepatic-differentiated CdH (CdH-Hep) and human hepatocytes (hPH) (Data is represented as the mean±SD).

FIG. 4C is a set of phase-contrast images of cholangiocytic induction of CdH (CdH-Chol).

FIG. 4D illustrates an immunofluorescence analysis result of cholangiocyte markers in cholangiocytic differentiation of CdH.

FIG. 4E is a quantitative comparison result of the relative gene expression levels of cholangiocyte markers for CdH, cholangiocytic differentiated CdH (CdH-Chol) and human hepatocytes (hPH).

FIG. 5A is a transmission electron microscope image of hepatic-differentiated CdH (CdH-Hep).

FIG. 5B is an assessment result of secreted albumin levels during hepatic induction.

FIG. 5C is a comparison result of expression of hepatocyte-specific genes between human hepatocytes (hPH), CdH and hepatic induced CdH (CdH-Hep) liver by microarray analysis.

FIG. 6A is a view illustrating the long term maintenance and stable state of CdH after passaging, the stable maintenance of CdH morphology according to passage number (Scale bars=100 μm) and an immunofluorescence analysis result of hepatic progenitor markers in CdH according to passage number (Scale bars=50 μm).

FIG. 6B illustrates a relative gene expression level of hepatic progenitor markers in CdH according to passage number (Data is represented as the mean±SD).

FIG. 6C is an immunofluorescence analysis result of hepatic marker proteins in hepatic-induced CdH (CdH-Hep) according to passage number.

FIG. 6D illustrates the comparison of gene expression aspect of hepatic markers in hepatic-induced CdH (CdH-Hep) with CdH (Data is represented as the mean±SD).

FIG. 7A illustrates a growth curve of CdH according to passage number (Data is represented as the mean±SD), and FIG. 7B illustrates a karyotype image of CdH according to passage number.

FIG. 8A is a schema of the experimental procedure for establishing human CdH and in vivo cell transplantation assay.

FIG. 8B is an immunofluorescence analysis result of hepatic marker proteins capable of confirming the hepatic differentiation ability of CdH transplanted in livers of NOD.Cg-Prkdc^{scid} (CdH transplanted in Il2rg^{tm1Wjl}/SzJ mice expresses hALB, HNF4a and is labeled with mCherry, and thus can be confirmed together on day 14, Scale bars=250 μm), and

FIG. 8C is an immunofluorescence analysis result of cholangiocytic marker proteins capable of confirming the cholangiocytes differentiation ability of CdH in livers of NOD.Cg-Prkdc^{scid} (CdH transplanted in Il2rg^{tm1Wjl}/SzJ mice expresses CK19, CK7, is labeled with mCherry, and thus can be confirmed together on day 14, Scale bars=250 μm).

### DETAILED DESCRIPTION OF EXEMPLARY EMBODIMENTS

Hereinafter, the configuration of the present invention will be specifically described.

The present invention relates to a medium composition for reprogramming human adult hepatocytes to hepatic progenitor cells, including HGF, A83-01, and CHIR99021.

Further, the present invention provides a method for reprogramming human adult hepatocytes to hepatic progenitor cells, wherein the method includes culturing human adult hepatocytes in a medium composition for reprogramming human adult hepatocytes to hepatic progenitor cells, including HGF, A83-01, and CHIR99021.

The present invention is characterized by providing a method for stably inducing human primary hepatocytes into chemically derived hepatic progenitors (CdH) by adding hepatocyte growth factor (H) as a hepatocyte growth factor, A83-01(A) as an ALK inhibitor, and CHIR99021(C) as a glycogen synthase kinase (GSK) 3 inhibitor to human primary hepatocytes.

The method for reprogramming human adult hepatocytes to hepatic progenitor cells of the present invention includes:

isolating primary hepatocytes from livers of normal and liver disease patients by a traditional two-step collagenase perfusion method; and

inducing the human primary hepatocytes into hepatic progenitor cells by culturing the human primary hepatocytes in a medium composition including HGF, A83-01, and CHIR99021.

In the isolating of the human primary hepatocytes, several days after treatment of human primary hepatocytes with two different chemical agents, homogenous polygonal cells appear and grow rapidly while coexisting human primary hepatocytes are dying. These chemically derived cells express hepatic and bile duct epithelial lineage genes and are stained with hepatic progenitor specific markers. According to a specific embodiment of the present invention, in hepatic progenitor cells, the expression of hepatic progenitor cells specific marker including albumin, AFP, SOX9, ITGA6, HNF6, EpCAM, FOXJ1, HNF1β, CK19, CD44, and CD90 was increased 1.5 times as compared to that in human adult hepatocytes.

According to a next generation sequencing (NGS) study, rapid growing hepatic progenitors (CdH) showed a gene expression pattern similar to that of human hepatoblasts. Chemically derived hepatic progenitors (CdH) from the human liver differentiate into hepatocytes and biliary epithelial cells, suggesting the nature of bipotent liver stem cells. Even after 10 passages, CdH doesn't lose its growth pattern and hepatic progenitor phenotype. CdH also engrafts and functions for several weeks in the immunosuppressed mouse model after transplantation through the intrasplenic route. Therefore, the CdH induction technique may be used as a therapeutic agent in the field of liver regenerative medicine.

In the present invention, HGF, A83-01, and CHIR99021 are included in a medium composition for reprogramming to hepatic progenitor cells using human adult hepatocytes, and HGF may be included at a concentration of 2 to 100 ng/mL in a human adult hepatocyte reprogramming medium composition. When the content is more than 100 ng/mL, apoptosis is affected, and when the content is less than 2 ng/mL, hepatic progenitor cells are not produced.

The A83-01 is known as an ALK inhibitor which is an inhibitor of TGF-beta signaling, and may be included at a concentration of 0.4 to 4 μM in a human adult hepatocyte reprogramming medium composition. When the content is more than 4 μM, apoptosis is induced, and when the content is less than 0.4 μM, the production of hepatic progenitor cells is insignificant.

The CHIR99021 is known as a small molecule inhibitor of glycogen synthase kinase (GSK)-3, and may be included at a concentration of 0.3 to 3 μM in the human adult hepatocyte reprogramming medium composition. When the content is 3 μM, apoptosis is induced, and when the content is less than 0.3 μM, the production of hepatic progenitor cells is insignificant.

The HGF, A83-01, and CHIR99021 may be added to a hepatocyte reprogramming medium. Therefore, the medium composition of the present invention may include a hepatocyte reprogramming medium and a growth medium.

The hepatocyte reprogramming medium may include all the media typically used in stem cell and progenitor cell cultures as well as in somatic cell culture in the corresponding field. A medium used for culture generally contains a carbon source, a nitrogen source, and trace element components. In a specific example of the present invention, a DMEM/F-12 medium supplemented with 0.1 μM dexamethasone, 10 mM nicotinamide, a 1% insulin-transferrin-selenium (ITS) premix, and penicillin/streptomycin/glutamine was used, and in addition, elements required for the culture of cells may be included without any limitation.

Human adult hepatocytes may be cultured in the medium composition for reprogramming human adult hepatocytes to hepatic progenitor cells of the present invention for 3 to 14 days and induced into hepatic progenitor cells. When it is out of the above range, hepatic progenitor cells may not be produced.

According to a specific embodiment of the present invention, when human adult hepatocytes are cultured in a DMEM/F-12 medium supplemented with 10% FBS, 0.1 μM dexamethasone, 10 mM nicotinamide, a 1% insulin-transferrin-selenium (ITS) premix, penicillin/streptomycin/glutamine, 20 ng/mL EGF, 20 ng/mL HGF, 4 μM A-83-01, and 3 μM CHIR99021 for 3 to 14 days, human adult hepatocytes were induced into hepatic progenitor cells, and the characteristics of hepatic progenitor cells, that is, the expression of hepatic progenitor cells specific markers began to be increased from day 7, and maintained for 14 days or more.

In the medium composition for reprogramming human adult hepatocytes to hepatic progenitor of the present invention, hepatic progenitor cells induced from human adult hepatocytes have an increased expression of hepatic progenitor cells specific markers as compared to that in human adult hepatocytes, and are novel cells having bipotent stemness differentiating into hepatocytes and biliary epithelial cells.

Accordingly, the present invention provides hepatic progenitor cells derived from human adult hepatocytes, having 1.5-fold increased expression of hepatic progenitor cells specific markers including albumin, AFP, SOX9, ITGA6, HNF6, EpCAM, FOXJ1, HNF1β, CK19, CD44, and CD90 compared to that in human adult hepatocytes, and having bipotent stemness differentiating into hepatocytes and biliary epithelial cells.

The hepatic progenitor cells may be provided as a source of hepatic precursor for liver regeneration due to bipotent stemness, and thus may be used as a therapeutic agent for liver disease.

Accordingly, the present invention provides a composition for treating liver disease, including the hepatic progenitor cells.

The composition for treating liver disease may be cell therapeutics.

Examples of the liver disease include chronic hepatitis, liver cirrhosis, metabolic liver disease, liver cancer, congenital hereditary liver disease, or the like, but are not limited thereto.

The composition for treating liver disease of the present invention may further include a pharmaceutically acceptable carrier.

The pharmaceutically acceptable carrier includes a carrier and a vehicle typically used in the medical field, and specific examples thereof include an ion exchange resin, alumina, aluminum stearate, lecithin, a serum protein (for example, human serum albumin), a buffer substance (for example, various phosphates, glycine, sorbic acid, potassium sorbate, partial glyceride mixture of saturated vegetable fatty acid), water, a salt or electrolyte (for example, protamine sulfate, disodium hydrogen phosphate, potassium hydrogen phosphate, sodium chloride, and zinc salts), colloidal silica, magnesium trisilicate, polyvinylpyrrolidone, cellulose-based substrate, polyethylene glycol, sodium carboxymethylcellulose, polyarylate, wax, polyethylene glycol, wool, or the like, but are not limited thereto.

In addition, the pharmaceutical composition of the present invention may additionally include a lubricant, a wetting agent, an emulsifier, a suspending agent, a preservative, or the like, in addition to the aforementioned ingredients.

As an aspect, the composition according to the present invention may be prepared into an aqueous solution for parenteral administration, and preferably, a buffer solution such as Hank's solution, Ringer's solution or a physically buffered saline solution may be used. In an aqueous injection suspension, a substrate capable of increasing the viscosity of the suspension, such as sodium carboxymethyl cellulose, sorbitol or dextran, may be added.

The composition of the present invention may be administered systemically or locally, and may be formulated into dosage forms suitable for known techniques for these administrations. For example, during the oral administration, the composition of the present invention may be administered while being mixed with an inert diluent or an edible carrier, sealed in a hard or soft gelatin capsule, or compressed in a tablet. For oral administration, the active compound may be incorporated with an excipient and used in the form of an ingestible tablet, a buccal tablet, a troche, a capsule, an elixir, a suspension, a syrup, a wafer, and the like.

Various dosage forms such as for injection and parenteral administration may be prepared and administered by techniques known in the art or commonly used techniques. For example, it may also be administered in a form suitable for intravenous injection, subcutaneous injection, intramuscular injection, intraperitoneal injection, transdermal administration, and the like, by being formulated into a solution immediately before administration in a saline or buffer solution.

An adequate administration amount of the composition of the present invention may vary depending on factors, such as formulation method, administration method, age, body weight, gender or disease condition of the patient, diet, administration time, administration route, excretion rate, and response sensitivity. For example, at least about 10⁴ to 10⁶ and typically, 1×10⁸ to 1×10¹⁰ cells may be infused intravenously or intraperitoneally into a 70 kg patient over roughly 60 to 120 minutes. For administration, in consideration of the overall health status and body weight of the individual, hepatic progenitor cells are administered at a rate determined by the LD-50 (or other methods of measuring toxicity) according to the cell type and the side-effects according to the cell type at various concentrations. Administration may be accomplished via single or divided doses.

The present invention also relates to a method for treating liver disease, wherein the method includes administering an effective amount of the composition for treating liver disease, including the hepatic progenitor cells to a subject in need thereof.

The subject may be a vertebrate, preferably, a mammal, for example, a dog, a cat, a rat, a human, or the like.

In the present invention, “treatment” refers to all the actions that suppress or alleviate or advantageously alter clinical situations related to a disease. Furthermore, the treatment may refer to increased survival as compared to an expected survival rate when the treatment is not received. The treatment simultaneously includes prophylactic means in addition to therapeutic means.

In the present specification, “effective amount” refers to an amount required to delay or completely stop the onset or progression of a specific disease to be treated. In the present invention, the composition may be administered in a pharmaceutically effective amount. It is obvious to a person skilled in the art that a suitable total usage amount per day may be determined by a doctor within the scope of sound medical judgment. For the purpose of the present invention, it is preferred that a specific therapeutically effective amount for a specific patient is differently applied depending on various factors including the type and extent of a response to be achieved, a specific composition including whether other formulations are used according to the case, the age, body weight, general health status, gender, and diet of the patient, the time of administration, the route of administration, the secretion rate of the composition, the period of treatment, a drug used in combination with or concurrently with the specific composition and similar factors well known in the medical field.

Hereinafter, the present invention will be described in detail through the Examples. However, the following Examples are only for exemplifying the present invention, and the content of the present invention is not limited by the following Examples.

### EXAMPLE 1

### Induction of Hepatic Progenitor Cells from Hepatocytes by Chemicals

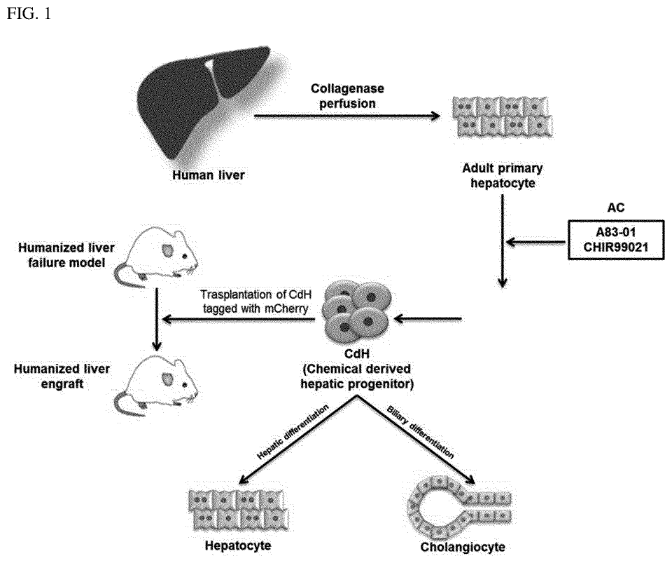

The inventors confirmed the reprogramming of hepatocytes by culturing human primary hepatocytes in various combinations based on a hepatocyte culture medium for direct reprogramming in order to investigate the combination of small molecules which may affect direct reprogramming (FIG. 1). The experimental method related to this is as follows.

### (Isolation of Human Primary Hepatocytes)

Primary hepatocytes were isolated from the normal livers of patients who had given informed consent using a two-step collagenase perfusion method (Table 1). Briefly, in step one, livers were perfused with solution A consisting of EDTA as a calcium chelating agent for 5 min at 37° C. In step two, livers were perfused with solution B consisting of a trypsin inhibitor and collagenase for 8 min at 37° C. Subsequently, livers were extracted and finely cut in a dish. Primary hepatocytes were washed with Williams' Medium E (Gibco, NY, USA) and treated with 25% Percoll (GE Healthcare, Bucks, UK). Primary hepatocytes thus obtained showed 80-90% viability.

TABLE 1
Clinical information about liver donors

| Donor | Age | Gender | Diagnosis | Surgery | Weight |
|---|---|---|---|---|---|
| 1 | 80 | Female | liver cancer | Right posterior | 4.02 g |
| | | | | sectionectomy | |
| 2 | 56 | Male | liver cancer | Left posterior | 5.95 g |
| | | | | sectionectomy | |
| 3 | 86 | Female | Colon cancer | Wedge resection | 0.74 g |
| | | | liver metastasis | | |
| 4 | 75 | Male | liver cancer | Laparoscopic wedge | 1.01 g |
| 5 | 54 | Female | Intrahepatic | Left lateral | 0.54 g |
| | | | stones | sectionectomy | |
| 6 | 64 | Male | Fatty liver | | 0.7 g |
| 7 | 31 | Female | Normal | | 0.71 g |

### (Establishment of Chemically Induced Hepatic Progenitor Cells)

After culturing with William's medium for 24 h, the human primary hepatocytes were cultured with DMEM F-12 (11965, Gibco, CA, USA) supplemented with 10 mM nicotinamide (Sigma-Aldrich, MO, USA), 1% penicillin/streptomycin (Gibco), 20 ng/mL HGF (Peprotech, NJ, USA), 20 ng/mL EGF (Peprotech), 4 μM A83-01(Gibco), and 3 μM CHIR99021 (StemCell Technologies) at 37° C. in a CO₂ incubator. The medium was changed every day.

### (Lentivirus Production)

mCherry was packaged by cotransfection with a psPAX2 lentiviral packaging plasmid and a pCMV-VSV-G plasmid in human embryonic kidney (HEK) 293T cells. Culture supernatants were harvested after 48 h and 72 h, and stored at −80° C. Lentiviral transduction of the mCherry was carried out in a culture medium supplemented with 8 μg/mL polybrene (Sigma-Aldrich).

(mRNA Isolation and RT-PCR Analysis)

Total RNAs were isolated using TRIzol Reagent (Gibco). 1 μg RNA samples were reverse transcribed with a Transcriptor First Strand cDNA Synthesis Kit (Roche, PA, USA), and real-time PCR was performed using 10 μL of qPCR PreMix (Dyne bio, Korea), 1 μL of cDNA and oligonucleotide primers (Table 2) with a CFX Connect Real-Time PCR Detection system (Bio-Rad, CA, USA). Reactions were analyzed in triplicate for each gene. The PCR cycle consisted of 40 cycles of 95° C. for 20 sec and 60° C. for 40 sec. Melt curves and melt peak data were obtained to characterize the PCR products.

TABLE 2
Oligonucleotide primers for real-time PCR

| | Forward Primer Sequence | Reverse Primer Sequence |
|---|---|---|
| Gene | (5′-3′) | (5′-3′) |
| AFP | AGACTGCTGCAGCCAAAGTGA | GTGGGATCGATGCTGGAGTG |
| SOX9 | GAGGAAGTCGGTGAAGAACG | ATCGAAGGTCTCGATGTTGG |
| EpCAM | GAACAATGATGGGCTTTATG | TGAGAATTCAGGTGCTTTTT |
| FOXJ1 | CCTGTCGGCCATCTACAAGT | AGACAGGTTGTGGCGGATT |
| CD44 | CATCTACCCCAGCAACCCTA | CTGTCTGTGCTGTCGGTGAT |
| ITGA6 | TCGCTGGGATCTTGATGCTTGC | TGAGCATGGATCTCAGCCTTGTGA |
| HNF6 | AGGGTGCTCTGCCGCTCCCAGG | CATGCTGCTAAGCGGAGCGCGGAC |
| HNF1β | GGGGCCCGCGTCCCAGCAAA | GGCCGTGGGCTTTGGAGGGGG |
| CK19 | TCCGAACCAAGTTTGAGACG | CCCTCAGCGTACTGATTTCC |
| CD90 | CTAGTGGACCAGAGCCTTCG | ACAGGGACATGAAATCCGTG |
| CK7 | GATAAAAGGCGCGGAGTGTC | GAAACCGCACCTTGTCGATG |
| CFTR | AGTTGCAGATGAGGTTGGGC | AAAGAGCTTCACCCTGTCGG |
| AE2 | GGGGAAAGTTGAGTTGGGAGA | CTGGCTCTGGCGTACAGAAA |
| AQPR1 | GGCCAGCGAGTTCAAGAAGAA | TCACACCATCAGCCAGGTCAT |
| Alb | CACAGAATCCTTGGRGAACAGG | ATGGAAGGTGAATGTTTCAGCA |
| HNF1α | AGAGAGGGGTGTCCCCATCA | CTTGTGCCGGAAGGCTTCTT |
| HNF4α | CCAAAACCCTCGTCGACATG | GCACATTCTCAAATTCCAGG |
| ASGR1 | CAGCAACTTCACAGCCAGCA | AGCTGGGACTCTAGCGACTT |
| CYP1A2 | CGGACAGCACTTCCCTGAGA | AGGCAGGTAGCGAAGGATGG |
| CYP2A6 | CAGCACTTCCTGAATGAG | AGGTGACTGGGAGGACTTGAGGC |
| CYP2C9 | CTACAGATAGGTATTAAGGACA | GCTTCATATCCATGCAGCACCAC |
| CYP3A4 | TTCAGCAAGAAGAACAAGGACAA | GGTTGAAGAAGTCCTCCTAAGC |
| GAPDH | TGCACCACCAACTGCTTAGC | GGCATGGACTGTGGTCATGAG |

### (Transmission Electron Microscope)

Tissue samples were fixed with paraformaldehyde and glutaraldehyde, and then fixed again with osmium tetroxide (OsO₄). Tissue samples fixed to resin, and then cut into ultrathin sections, stained with uranyl acetate and lead citrate, and pictures were taken.

### (Time Lapse Imaging)

Time lapse imaging was performed using a JuLi stage system (NanoEnTek). The cell culture dish was placed on a microscope, and then the production of hepatic progenitor cells was captured once per 60 minutes while culturing cells at 5% CO₂ and 37° C. For data analysis, JuLi stage software v1.0 was used.

### (FACS Sorting)

To isolate mCherry fluorescence expressing-CdH cells, the samples were resuspended in PBS containing 10% FBS and 2 mM EDTA and sorted with FACS Aria□ (BD Biosciences) equipped with Turbo Sort for high speed sorting. After sorting, viability was determined by trypan blue, and was typically 90% or more.

### (Immunohistochemistry)

4 μm sections were deparaffinized by washing three times with xylene for 5 min each time. The CdH was rehydrated with ethanol, and finally washed with distilled water. After washing, antigens were retrieved after autoclaving the slides in pH 6.0 and 10 mM sodium citric acid buffer and exposing the antigens. The slides were incubated in distilled water for 5 min, supplemented with primary antibodies at 4° C. overnight, and then put into a chamber where constant humidity was maintained. The next day, the sections were washed three times with a staining solution which is PBS supplemented with 1% FBS for 5 min, then incubated with secondary antibodies for 1 h and 30 min at room temperature. Antibodies are shown in Table 3. Nuclei were counterstained with Hoechst 33342 (1:10000, Molecular Probes) dissolved in PBS. After constant-temperature incubation with secondary antibodies, the slides were washed with running tap water, dehydrated, cleared and mounted. Samples were imaged by a TCS SP5 confocal microscope (Leica).

TABLE 3
Antibodies list

| Protein | Company | Product number |
|---|---|---|
| Albumin | Abcam | ab19194 |
| CK19 | Santa Cruz Biotechnology | sc-376126 |
| OV-6 | Santa Cruz Biotechnology | sc-101863 |
| EpCAM | Abcam | ab32392 |
| CD44 | Cell Signaling | 5640S |
| CD90 | Abcam | ab92574 |
| AFP | Abcam | ab3980 |
| SOX9 | Abcam | ab185966 |
| HNF4α | Santa Cruz Biotechnology | sc-8987 |
| CK18 | Abcam | ab668 |
| CYP3A4 | Santa Cruz Biotechnology | sc-27639 |
| AE2 | Abcam | ab42687 |
| CK7 | Abcam | ab9021 |
| CFTR | Almone | Acl006 |
| mCherry | Abcam | ab167453 |

### (Microarray Analysis)

Total RNA samples were extracted with TRIzol Reagent (Gibco), and provided to LAS, Inc (LAS, Korea) for analysis. Briefly, Mouse Ref-8v3 Sentrix bead chips (Illumina, San Diego, Calif., USA) were used, and normalized by the quantile normalization method across all samples. Arrays were scanned using an Illumina Bead Array Reader Confocal Scanner. The microarray data was deposited in the NCBI Gene Expression Omnibus. Genes were clustered with Cluster 3.0, and heat maps were generated with Tree View 3.0.

### (Hepatic Induction)

For hepatocyte differentiation, CdH was cultured on a collagen type I-coated dish at 4×10⁵˜5×10⁴ cells/cm² according to a hepatocyte maturation method of a previously published paper. After culturing with a CdH medium for 2 days, the medium was replaced with a hepatic induction medium consisting of 200 ng/mL oncostatin M (R&D Systems), and 10⁻⁷ mol/L Dex (Sigma-Aldrich), and the hepatic induction medium were provided every 2 days. After culture for 6 days, the medium was replaced with a 1:7 mixture of Matrigel (BD Biosciences) and a hepatic induction medium. On day 8, the mixture was removed by washing with Hank's Balanced Salt Solution (HBSS).

### (Cholangiocyte Induction)

For the generation of cholangiocytes, a three-dimensional culture system using collagen type I (BD) was used according to the manufacturer's instruction. In brief, 800 μl of Collagen type 1, 100 μl of 10×PBS, 20 μl of 1 N NaOH, and 80 μl of H₂O were mixed on ice. This mixture was mixed with an equal volume of 1×10⁵ cloned CdH suspended in a cholangiocytic differentiation medium (CDM) (DMEM/F12 medium supplemented with 10% fetal bovine serum and 20 ng/ml HGF [BD]). The cell suspension was transferred into a 6-well plate and left at 37° C. for 30 min. After the gel was formed, the CDM was gently added to the gel.

### (Periodic Acid Schiff (PAS) Staining and Indocyanine Green Staining)

The induced cells were harvested from the top of the Matrigel gel by dispase and then were used for periodic acid Schiff (PAS) staining and indocyanine green (ICG) staining. Periodic acid-Schiff (PAS) staining was performed to detect glycogen using a PAS kit (abcam) with or without a salivary diastase pretreatment at 37° C. for 15 min. Hematoxylin and eosin (HE) staining was performed using a standard procedure. Indocyanine green (ICG, Dai-ichi Pharmaceutical) staining was performed on the induced cells and the cells were cultured at 37° C. for 15 min. After the cells were rinsed with PBS, the ICG uptake was observed by phase-contrast microscopy.

### (Enzyme-Linked Immunosorbent Assay (ELISA))

Alb secretion assay was carried out with a Human Albumin ELISA Kit (Bethyl Laboratories). To observe alb secretion ability, the culture conditioned medium was collected every 2 days for a hepatic induction time. An assay procedure followed the protocol of the Human Albumin ELISA Kit.

### (Karyotyping)

Karyotyping of hepatic progenitor cells (CdH) was performed by EONE Laboratories.

### (Experimental Animal Model)

NOD.Cg-Prkdcscid Il2rg^{tm1Wjl}/SzJ mice were purchased from the Jackson Laboratory and housed under specific pathogen-free conditions in accordance with the Principles of Laboratory Animal Care and the Guide for the Use of Laboratory Animals of the Samsung Biomedical Research Institute. Twenty-four hours after intraperitoneal injection of Jo2 antibody/PBS (BD Pharmingen, CA, USA) at 0.1 to 2.2 mg/kg body weight, CdH and human hepatocyte direct reprogrammed hepatic progenitor cells (1×10⁶ cells/100 μl) were transplanted. After implantation into the spleen of NSG mice, the mice received 100 mg/L ciprofloxacin (CJ Pharma, Korea) in their drinking water to prevent infection. At termination, mouse liver tissue was immediately fixed in 10% formalin.

### (Statistical Analysis)

Quantitative data is expressed as means±standard deviations (SD) with inferential statistics (p values). Statistical significance was evaluated by two-tailed t-tests with significance set at *P<0.05, **P<0.01, ***P<0.001.

### Experimental Example 1

### Generation of Hepatic Progenitor Cells by Small Molecules

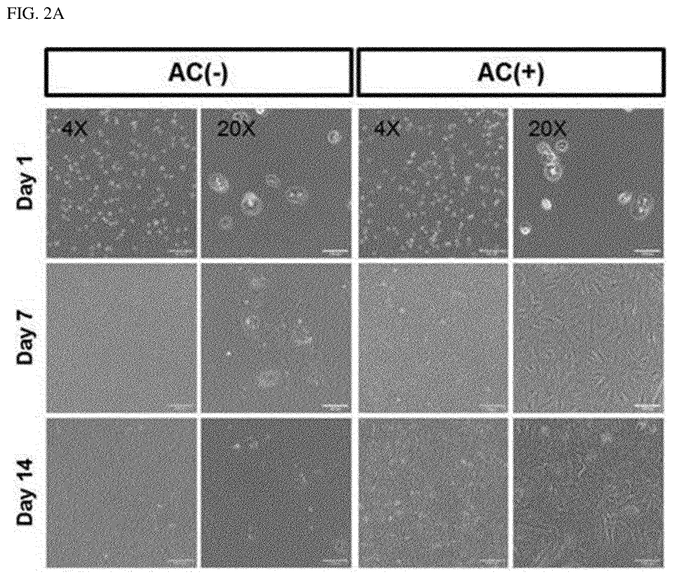

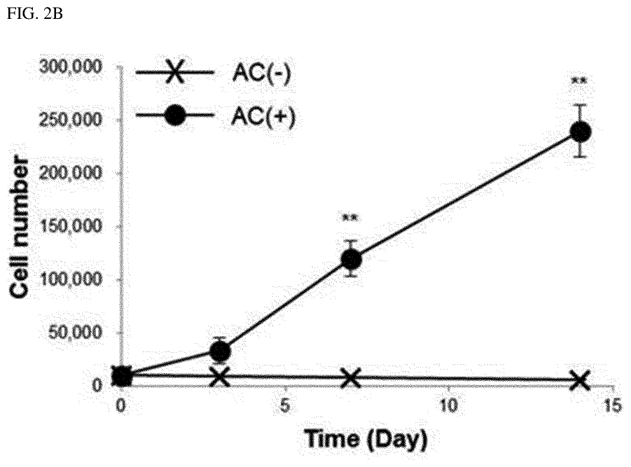

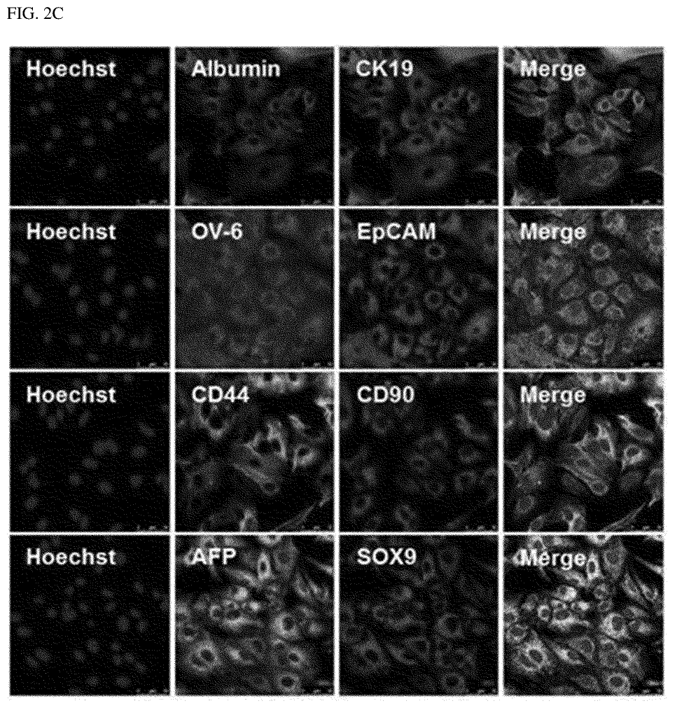

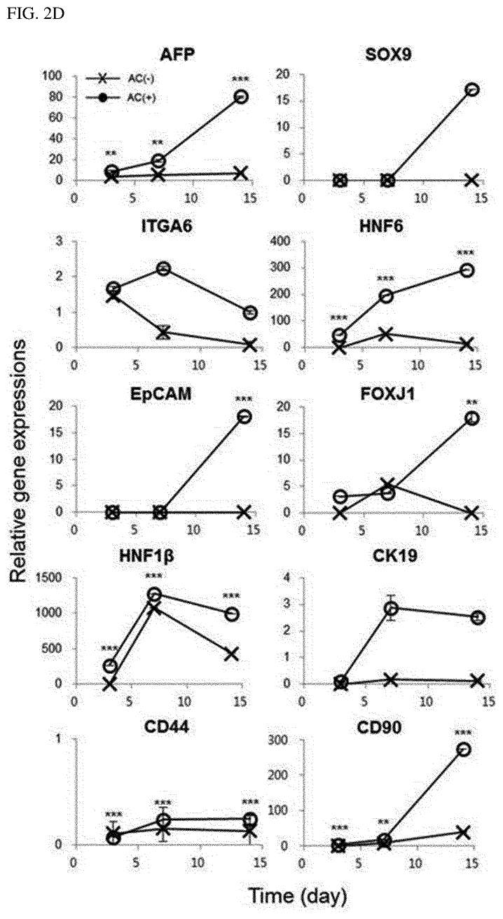

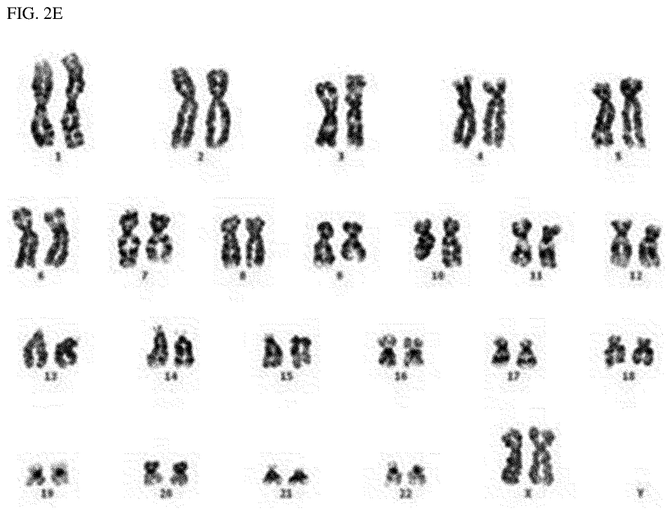

As described above, as a result of investigating the combination of small molecules capable of affecting direct reprogramming of human adult hepatocytes, hepatocytes which had not been treated with small molecules were changed into fibrous forms without proliferation in vitro, and hepatocytes cultured under an AC condition (combination of HGF, A83-01, and CHIR99021) proliferated in an epithelial form after 7 days. Hepatocytes cultured under the AC condition did not show any significant change in growth rate until day 3, but thereafter showed a rapid growth rate. The proliferation capacity for 2 weeks was 22.6- to 26.4-fold and a single cell proliferation ability was conformed (FIGS. 2A and 2B).

To investigate the characteristics of hepatic progenitor cells at the protein level of CdH, immunocytochemistry was conducted to visualize the amount of various hepatic progenitor markers. Images showed that various progenitor markers, such as Albumin, CK19, OV-6, EpCAM, CD44, CD90, AFP, and SOX9 were stably expressed (FIG. 2C). These results suggested that human hepatocytes were reprogrammed to and turned into progenitor cells again.

As a result of verifying the point of conversion from hepatocyte to CdH by qRT-PCR, the expression of AFP, SOX9, ITGA6, HNF6, EpCAM, FOXJ1, HNF1β, CK19, CD44, and CD90 was found to be elevated between 7 and 14 days (FIG. 2D). Therefore, human hepatocytes differentiated into CdH when cultured for 7 days to 14 days under the AC condition, and the hepatic progenitor cell characteristic of CdH started to appear from day 7 and was maintained until day 14.

Further, in 20 analyzed metaphase cells, no clonal chromosomal abnormalities were observed and all showed normal karyotypes (FIG. 2E).

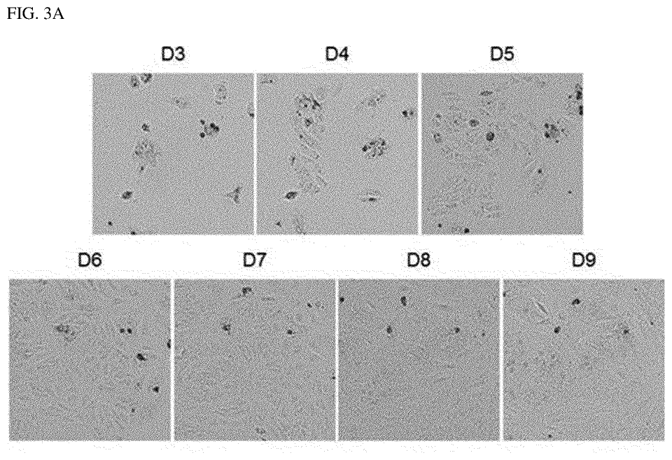

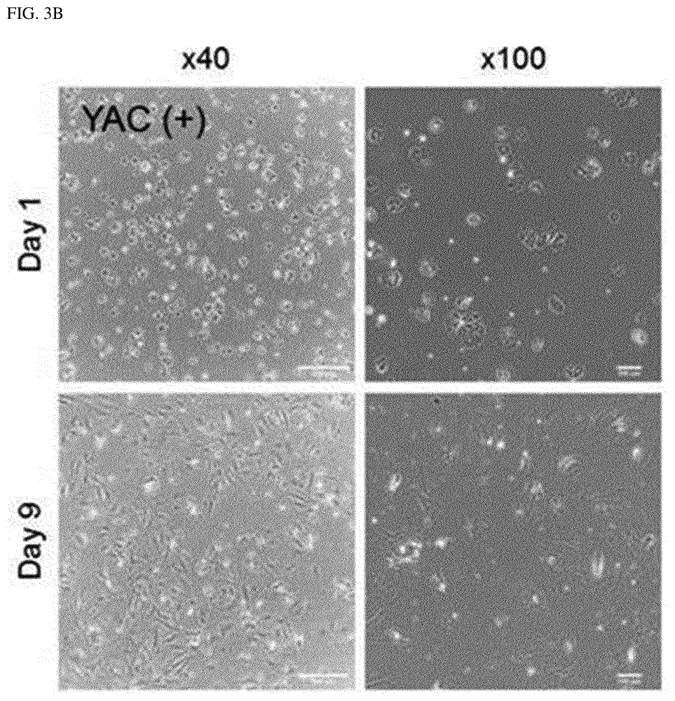

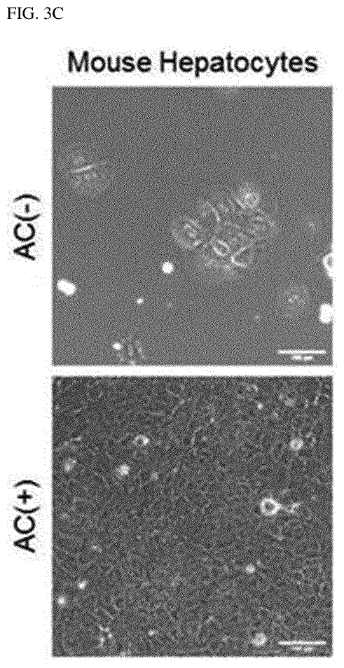

As a result of time lapse imaging of human hepatocytes cultured under the AC condition for 9 days, hepatocytes cultured under the AC condition showed rapid proliferation after 3 days (FIG. 3A).

To confirm whether human hepatocytes were converted into hepatic progenitor cells under YAC conditions found in mice, human hepatocytes were cultured in a medium containing YAC, and the medium was exchanged every two days.

As a result, human hepatocytes cultured under YAC conditions for 9 days were fibrotic and killed without being formed as CdH (FIG. 3B).

Further, conversely, to confirm whether mouse hepatocytes could be changed into CdH under an AC(+) condition, mouse primary hepatocytes were isolated by a two-step collagenase perfusion method and cultured under the AC(+) or AC(−) condition. As a result, it was confirmed that mouse primary hepatocytes cultured under the AC(+) condition for 7 days differentiated into CdH (FIG. 3C). Mouse CdH showed higher level of hepatic progenitor markers compared to primary hepatocytes (not shown).

The conversion from human hepatocytes to CdH occurred under the AC(+) condition, but didn't happen under YAC conditions. Therefore, in order to determine the major factor when human hepatocyte converted into CdH, an investigation was made by varying YAC culture conditions along with human hepatocytes.

As a result, CdH morphology was observed under HGF(+) and AC(+) conditions (FIG. 3D). However, under a HGF(−) condition, the growth rate of CdH decreased and eventually a proliferation capacity was lost. Thus, the combination of HGF and AC appears to play an important role in the conversion of human hepatocytes into hepatic progenitor cells, and HGF seems to be involved in the maintenance and proliferation of hepatic progenitor cells.

### Experimental Example 2

### Bipotent Differentiation of CdH into Hepatocytes and Bile Duct Epithelium

To identify the most important bipotent feature of hepatic progenitor cells that can differentiate into hepatocytes and cholangiocytes, a previously reported hepatocyte and cholangiocyte differentiation protocol was used.

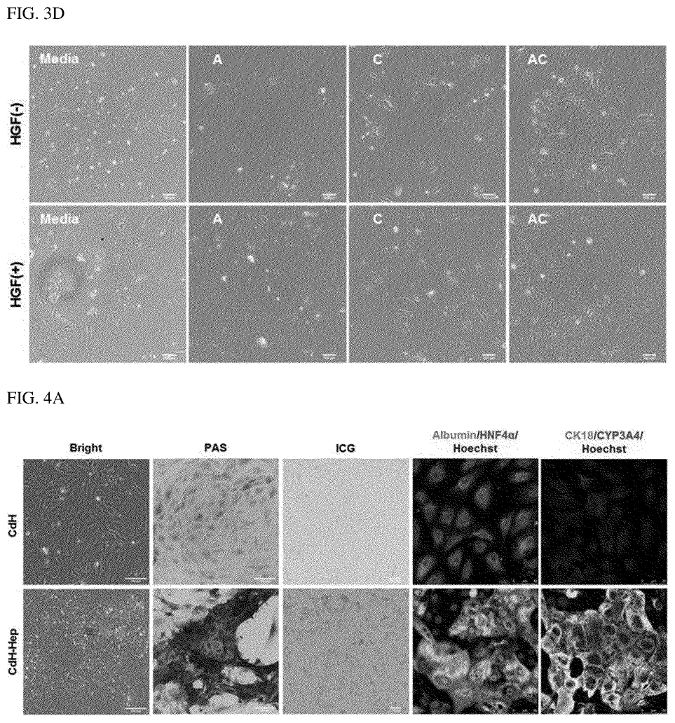

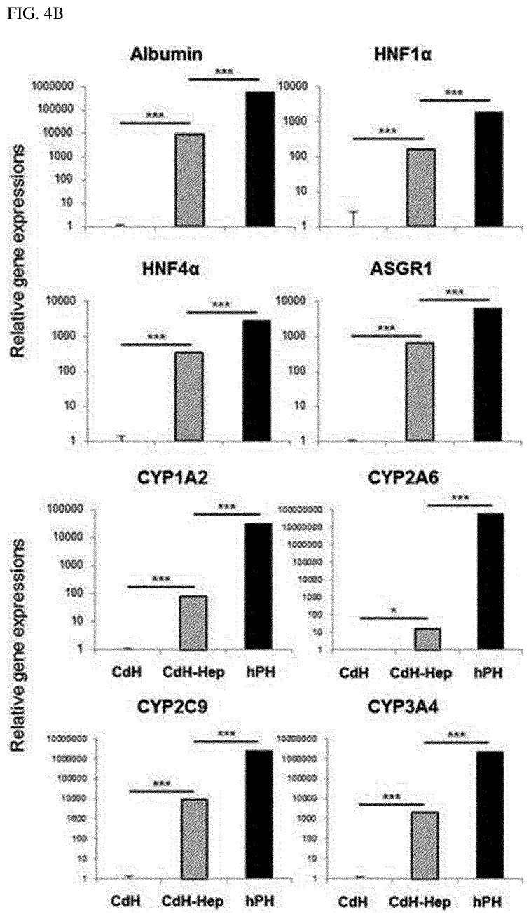

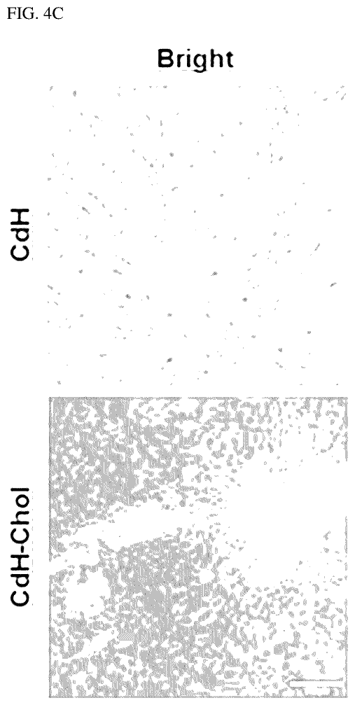

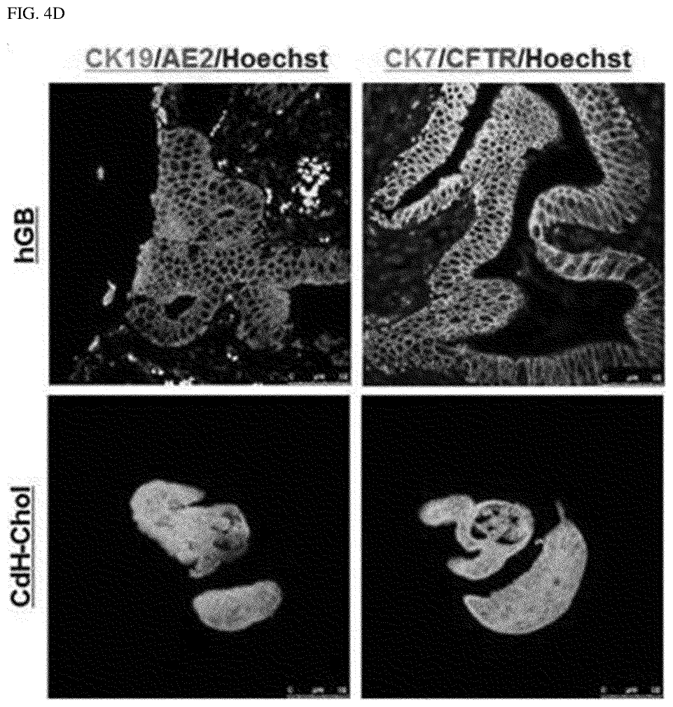

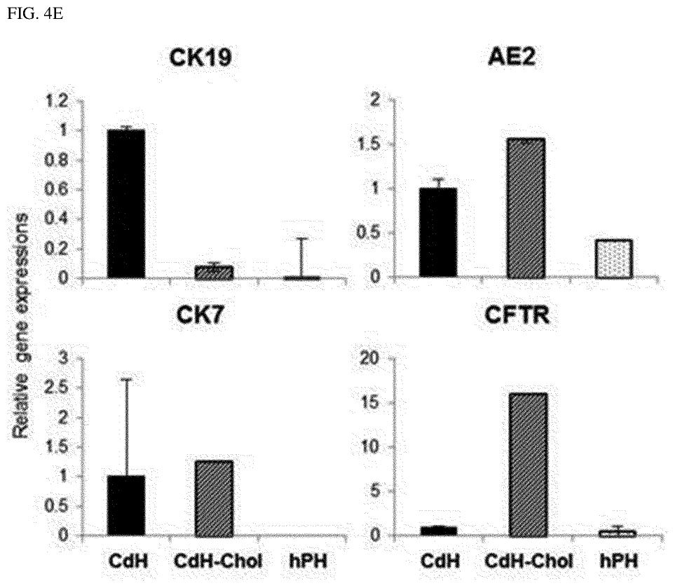

Hepatic-induced CdH showed typical hepatocytes having a bile canaliculi structure. Furthermore, glycogen storage, indocyanine green uptake and albumin secretion results indicated that induced CdH obtained the functional characteristics of a hepatocyte. Further, results of immunostaining show that the expression of hepatocyte specific proteins, Albumin, HNF4α, CK18, and CYP3A4 was expressed in CdH-induced hepatocyte differentiation (FIG. 4A). In addition, mRNA expression of hepatic genes such as Albumin, HNF1α, HNF4α, ASGR1 and CYP genes related to liver function was significantly higher than that of CdH that did not induce hepatocyte differentiation (FIG. 4B).

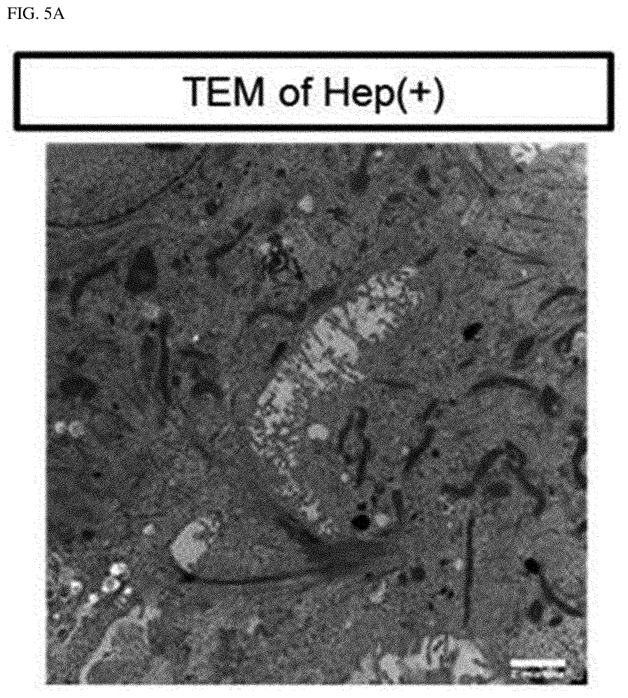

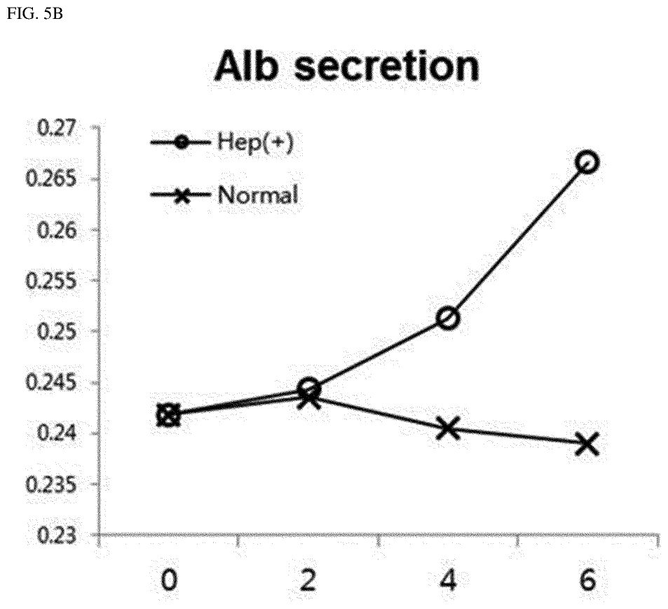

Furthermore, as a result of evaluating the value of albumin during the hepatocytic induction, the secretion of albumin of CdH was rarely changed, but the albumin secretion of hepatic-differentiated CdH was slowly increased (FIG. 5B).

As a result of microarray analysis, hepatic-differentiated CdH (CdH-Hep) showed a completely different global gene expression pattern from CdH before differentiation, and the pattern was very similar to that of hepatocytes isolated and extracted from a human liver (FIG. 5C).

When CdH was cultured in a three-dimensional (3D) type I collagen gel culture system, CdH differentiated into cholangiocyte cells with representative branching structures (FIG. 4C), and cholangiocyte markers CK19, AE2, CK2, and CFTR were identified at 9 days after induction (FIG. 4D). In many CdH-derived cells with branching structures, functional cholangiocytic markers CK19, CK7, AE2, and CFTR were observed along with human gallbladder (FIG. 4D), and the expression of the marker genes was also increased (FIG. 4E). This suggests that CdH differentiates into cholangiocyte-like cells.

### Experimental Example 3

### Long-Term Maintenance of CdH and Stable Condition After Passaging

To confirm that CdH maintains hepatic progenitor characteristics even in long-term passaging, the characteristics of CdH at passage 5 and passage 10 were identified and the passaging of CdH was stable to at least 15 passages.

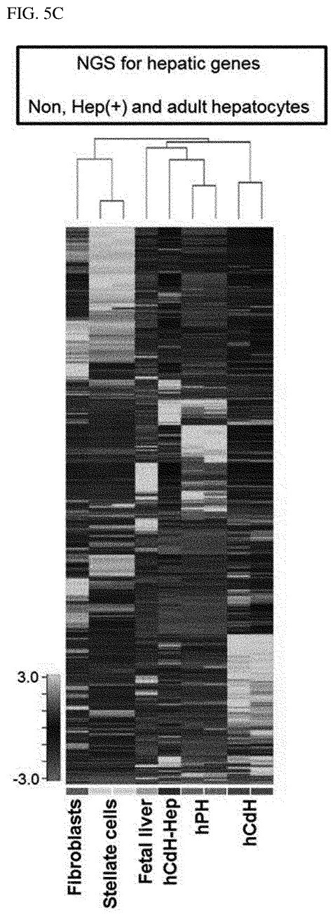

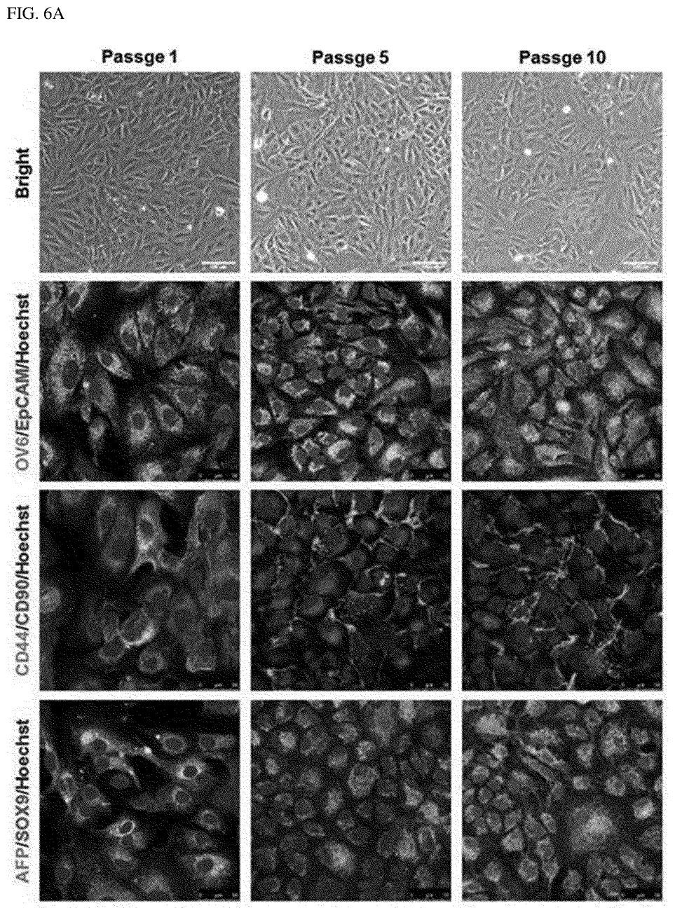

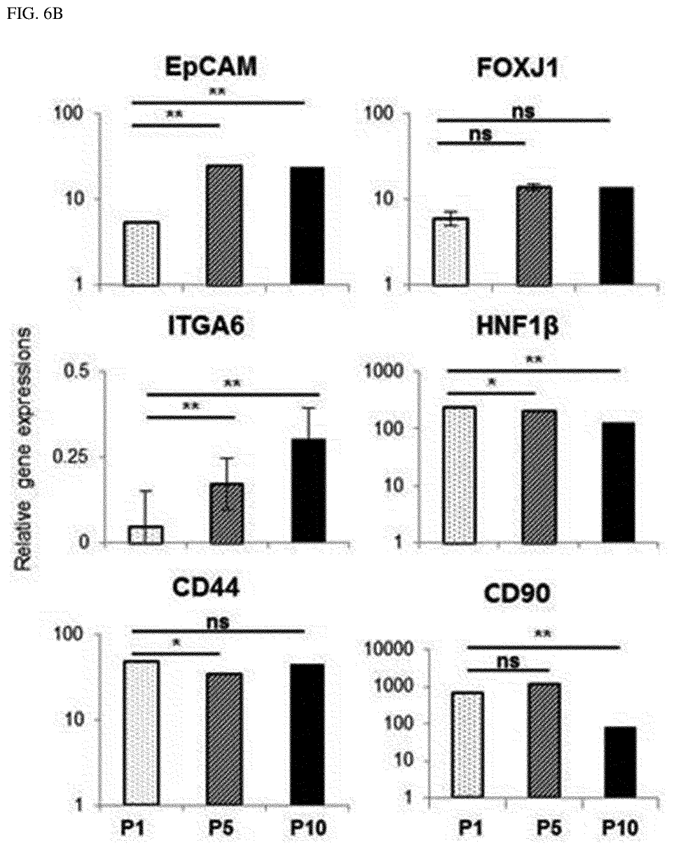

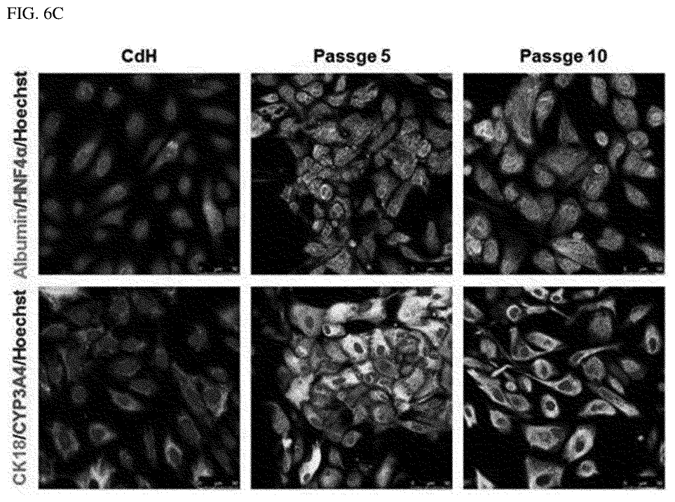

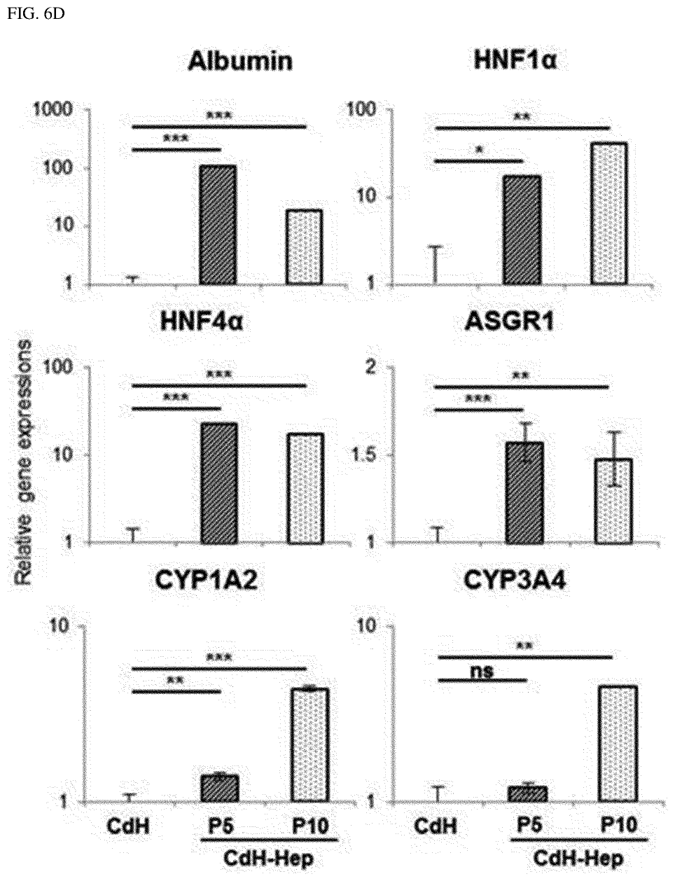

Morphologically, CdH retained their homogenous polygonal form up to passage 10 or more (FIG. 6A).

To verify that CdH was well characterized as a hepatic progenitor even after passaging, hepatic progenitor specific proteins were immunostained after 10 passages. The expression of hepatic progenitor cells specific proteins such as OV6, EpCAM, CD44, CD90, AFP and SOX9 was almost similar at passage 1, 5 and 10 (FIG. 6A).

Expression of hepatic progenitor genes, EpCAM, FOXJ1, ITGA6, HNF1β, CD44, and CD90 were compared using qRT-PCR analysis to identify the expression of hepatic progenitor genes at the mRNA level in long-term passaging. As a result, it was confirmed that progenitor markers were consistently expressed even after 10 passages (FIG. 6B).

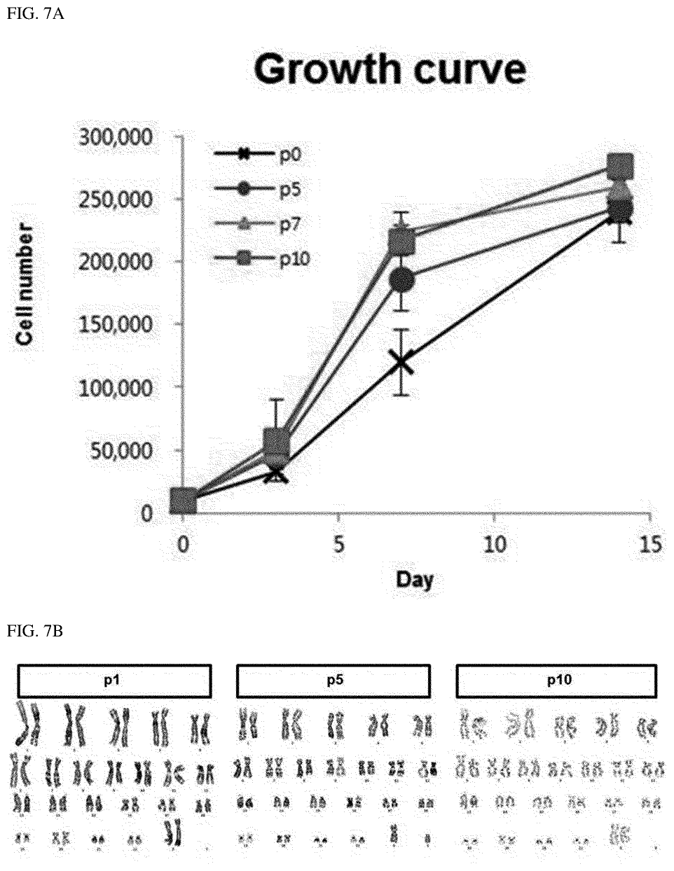

Further, the proliferative capacity of CdH was almost a similar rate after passaging and even grew faster as the passage passed (FIG. 7A).

In order to investigate the defect status of chromosomes in CdH, karyotyping of each of 20 metaphases cells at passage 1, 5, and 10 was performed. In the 20 metaphases cells of passage 1, 5, and 10, no clonal chromosomal anomalies were observed and all showed normal karyotypes (FIG. 7B). These results mean that CdH stably undergoes passaging and propagates without modification to chromosomal translocation.

Next, as a result of investigating whether CdH stably had a hepatic differentiation ability in long-term passaging, it was confirmed that CdH subjected to passage 5 and 10 stably differentiated into hepatocytes (FIG. 6C). Hepatocyte specific proteins Albumin, HNF4α, CK18, and CYP3A4 were expressed well in CdH-induced hepatocytes at the protein level, indicating that a hepatocyte differentiation ability was maintained well in long-term passaging (FIG. 6C). According to passage number, mRNA expression levels of various genes related to liver function fluctuated, but the expression of these genes were increased by maintaining a hepatic differentiation ability, and P450-related genes were found to increase with passaging (FIG. 6D). These results suggest that CdH can be stably passaged and has an ability of differentiation into hepatocytes in long-term passaging.

### Experimental Example 4

### In Vivo Transplantation and Engraftment of CdH

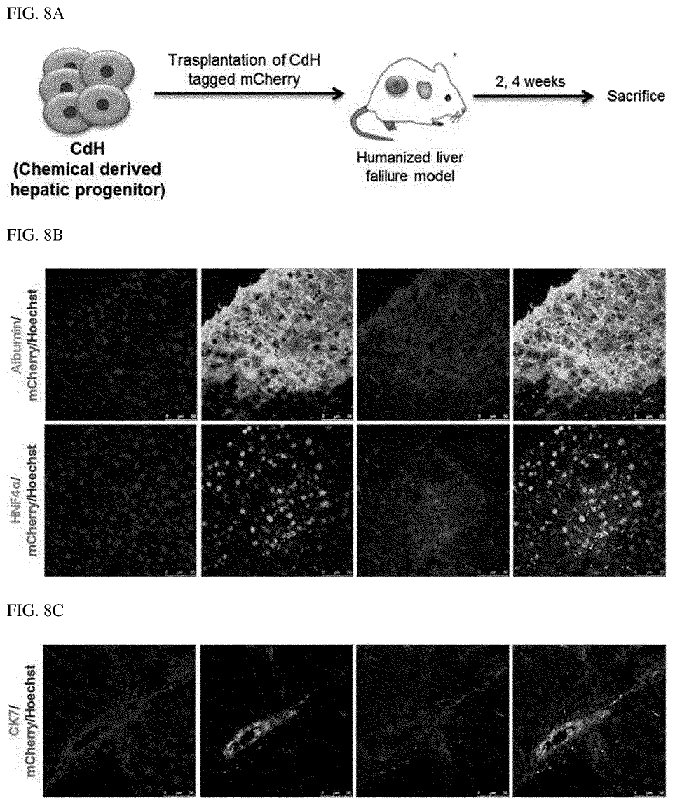

CdH was injected into Jo2 antibody-treated NSG (NOD.Cg-Prkdc^{scid} Il2rg^{tm1Wjl}/SzJ) mice (FIG. 8A). It was confirmed that in the livers of NSG mice transplanted with CdH, mCherry-expressing CdH was engrafted or differentiated into hepatocytes through the staining of albumin and Hnf4a proteins (FIG. 8B), and it was confirmed that through staining of cholangiocytic markers CK19 and CK7 proteins, mCherry-expressing CdH also differentiated into cholangiocytes (FIG. 8C).

From the results, it can be suggested that the substantial lack of supply of hepatocytes due to donor shortage and a lack of proliferative capacity of primary hepatocytes currently remaining as a major problem may be solved by CdH which is a hepatic stem cell with a self-renewal capacity.

In this context, the present invention is the first report of chemically reprogrammed human hepatic progenitor cells (CdH) of a robust culture system for the generation of significant numbers of hepatocytes and biliary epithelial cells, and upon transplantation into the liver of immunodeficient mice, CdH acquires the differentiated properties and growth pattern of adult hepatocytes without the development of teratomas, and thus may provide a valuable source of hepatic precursors for liver regeneration.

The present invention can be applied to the field of treating liver disease.

What is claimed is:

1. A medium composition for reprogramming human adult hepatocytes to hepatic progenitor cells, comprising HGF, A83-01, and CHIR99021.

2. The medium composition of claim 1, wherein hepatic progenitor cells have 1.5-fold increased expression of hepatic progenitor cells specific marker comprising albumin, AFP, SOX9, ITGA6, HNF6, EpCAM, FOXJ1, HNF1β, CK19, CD44, and CD90 compared to that in human adult hepatocytes, and have bipotent stemness differentiating into hepatocytes and biliary epithelial cells.

3. A method for reprogramming human adult hepatocytes to hepatic progenitor cells, comprising: culturing human adult hepatocytes in a medium composition for reprogramming human adult hepatocytes to hepatic progenitor cells, comprising HGF, A83-01, and CHIR99021.

4. The method of claim 3, wherein HGF is comprised at a concentration of 2 to 100 ng/mL in the human adult hepatocyte reprogramming medium composition.

5. The method of claim 3, wherein A83-01 is comprised at a concentration of 0.4 to 4 μM in the human adult hepatocyte reprogramming medium composition.

6. The method of claim 3, wherein CHIR99021 is comprised at a concentration of 0.3 to 3 μM in the human adult hepatocyte reprogramming medium composition

7. The method of claim 3, wherein the human adult hepatocytes are cultured for 3 to 14 days.

8. Hepatic progenitor cells derived from human adult hepatocytes, having 1.5-fold increased expression of hepatic progenitor cells specific marker comprising albumin, AFP, SOX9, ITGA6, HNF6, EpCAM, FOXJ1, HNF1β, CK19, CD44, and CD90 compared to that in human adult hepatocytes, and having bipotent stemness differentiating into hepatocytes and biliary epithelial cells.

9. A composition for treating liver disease, comprising the hepatic progenitor cells of claim 8.

10. The composition for treating liver disease of claim 9, wherein the liver disease is any one of chronic hepatitis, liver cirrhosis, metabolic liver disease, liver cancer, or congenital hereditary liver disease.

11. A method for treating liver disease, comprising:
administering an effective amount of a composition for treating liver disease comprising hepatic progenitor cells, to a subject in need thereof,
wherein the hepatic progenitor cells are derived from human adult hepatocytes, have 1.5-fold increased expression of hepatic progenitor cells specific marker including albumin, AFP, SOX9, ITGA6, HNF6, EpCAM, FOXJ1, HNF1β, CK19, CD44, and CD90 compared to that in human adult hepatocytes, and have bipotent stemness differentiating into hepatocytes and biliary epithelial cells.

12. The method of claim 11, wherein the liver disease is any one of chronic hepatitis, liver cirrhosis, metabolic liver disease, liver cancer, or congenital hereditary liver disease.
